(19) United States
(12) Patent Application Publication
(10) Pub. No.: US 2003/0035836 A1
(43) Pub. Date: Feb. 20, 2003

(54) Oral controlled release pharmaceutical composition for once-a-day therapy for the treatment and prophylaxis of cardiac and circulatory diseases

(76) Inventors: Dilip Shantilal Shanghvi, Mumbai (IN); Bala Ramesha R. Chary, Warangal (IN); Ziauddin Z. Tyebji, Maharashtra (IN)

Correspondence Address: PENDORF & CUTLIFF, Post Office Box: 20445, Tampa, FL, 33622-0445, US

(21) Appl. No.: 10/146,670

(22) Filed: Aug. 19, 2002

(30) Foreign Application Priority Data
May 17, 2001 (IN) 464/MUM/2001

(51) Int. Cl. A61K 31/407; A61K 9/22

(52) U.S. Cl. 424/468; 514/411

(57) ABSTRACT

The present invention relates to an oral controlled release pharmaceutical composition for once-a-day therapy for the treatment and prophylaxis of cardiac and circulatory diseases comprising carvedilol or its pharmaceutically acceptable salt or ester and release rate controlling excipients, wherein the said composition is adapted to release the carvedilol in a controlled manner so as to provide control over carvedilol plasma levels, such that the ratio of peak plasma levels to the plasma levels at 24 hours after administration, and the mean residence time of carvedilol, are within a desirable range for said once-a-day therapy for the treatment and prophylaxis of cardiac and circulatory diseases.

### BACKGROUND OF THE INVENTION

1. Field of the Invention

The present invention relates to an oral controlled release pharmaceutical composition for once-a-day therapy for the treatment and prophylaxis of cardiac and circulatory diseases in humans and to a process for the preparation of said composition.

More particularly, the present invention relates to an oral controlled release pharmaceutical composition that releases carvedilol in a controlled manner so as to provide control over carvedilol plasma levels, such that the ratio of peak plasma levels to the plasma levels at 24 hours after administration, and the mean residence time of carvedilol, are within a desirable range for said once-a-day therapy for the treatment and prophylaxis of cardiac and circulatory diseases.

The present invention also relates to a method of obtaining desired control over carvedilol plasma levels for once-a-day therapy for the treatment and prophylaxis of cardiac and circulatory diseases in humans, said method consisting of orally administering to human subjects said oral controlled release pharmaceutical composition.

The term cardiac and circulatory diseases as herein described includes hypertension, congestive heart failure, angina pectoris, left ventricular hypertrophy, arrhythmias, myocardial infarction, reflex tachycardia, ischaemic heart disease, atheromatosis, hypertension associated with diabetes mellitus, stroke and renal failure.

2. Description of the Related Art

Carvedilol, 1-(9H-carbazol-4-yloxy)-3-[[2-(2-methoxyphenoxy)ethyl]amino]-2-propanol, described in U.S. Pat. No. 4,503,067, is a competitive non-selective β-adrenergic blocking agent with α₁-blocking activity. The β-adrenergic blocking activity prevents reflex tachycardia in hypertension and the α₁-blocking activity causes vasodilation. The β-adrenergic blocking activity resides in the S(−) enantiomer, while the R(+) enantiomer possesses α₁-blocking activity. The drug is used as its racemic mixture so that both the enantiomers act together to exert the pharmacological effect of carvedilol.

Carvedilol is a novel multiple action drug useful in the treatment of mild to moderate hypertension and congestive heart failure. The drug is also known to act as a calcium channel blocker at high doses. The antihypertensive effect of carvedilol is mediated primarily by decreasing total peripheral vascular resistance without causing the concomitant reflex changes in heart rate commonly associated with other antihypertensive agents. Carvedilol also markedly reduces infarct size, possibly as a consequence of its antioxidant action in attenuating oxygen free radical-initiated lipid peroxidation, thereby leading to cardioprotection. For hypertension, the recommended starting dose for carvedilol is 6.25 mg given twice daily, which is increased after 7-14 days, if tolerated, to 12.5 mg twice daily, this dose being further increased to 25 mg twice daily, if tolerated and needed. The total daily dose should not exceed 50 mg. For congestive cardiac failure, the recommended starting dose is 3.125 mg given twice daily, which is increased to 6.25 mg twice daily after two weeks, if tolerated. The maximum recommended dose is 25 mg twice daily in patients weighing less than 85 kg and 50 mg twice daily in patients weighing more than 85 kg.

Carvedilol undergoes considerable first pass metabolism after oral administration and as a result has a low absolute bioavailability of 25%. Carvedilol is metabolized primarily by aromatic ring oxidation and glucuronidation. The oxidative metabolites are further metabolized by conjugation via glucuronidation and sulfation. Demethylation and hydroxylation at the phenol ring produce three active metabolites with β-blocking activity. Plasma concentrations of the active metabolites are about one-tenth of that for carvedilol and the pharmacokinetics is similar to carvedilol. Controlled release and delayed release formulations of carvedilol can give rise to once daily formulations which are able to extend the duration of action of carvedilol and thus improve the bioavailability of the drug. Hence, it would be advantageous to formulate a modified release composition for carvedilol, wherein the modified release may be delayed release, sustained release or controlled release.

Several adverse effects encountered in medical therapy are related to a peak in plasma concentration, often occurring a few hours after administration of a dose. Very rapid initial rate of release of carvedilol results in higher peak plasma levels and therefore, more adverse effects. On the other hand, if the carvedilol is released too slowly from the tablets, then incomplete absorption occurs. In the present invention, the ratio of the peak plasma levels to the plasma levels at 24 hours after administration is within a desirable range. A higher ratio of maximum plasma concentration of carvedilol to the plasma concentration at 24 hours after oral administration indicates a poorer control and faster release, while a smaller ratio indicates a control on the release rate over a prolonged duration. A higher ratio for a smaller dose of 12.5 mg daily may also mean that effective plasma levels of carvedilol may not be available at 24 hours after administration, whereas if the ratio is too small then the effective plasma levels of carvedilol may not be reached at all. On the other hand an optimum design of an oral controlled release composition for once-a-day therapy for the treatment and prophylaxis of cardiac and circulatory diseases requires that the composition provide a control on the plasma levels such that the mean residence time (i.e. the mean time that a drug spends in the body) is within a desirable range for said once-a-day therapy for the treatment and prophylaxis of cardiac and circulatory diseases.

PCT application WO 9924017 ('017) claims a matrix formulation comprising carvedilol in an oral dosage unit form. The systems exemplified include three different types: the first is a matrix tablet containing hydroxypropyl methylcellulose and Carbomer 934P as rate controlling excipients; the second is an immediate release core coated with an enteric polymer or a controlled release polymer; and the third is beads that are coated with glycerylmonostearate and glyceryldistearate. The application does not suggest to the person skilled in the art the manner in which the composition could be optimized and tested using a suitable test performance criteria such as release or dissolution profile, so as to provide control over carvedilol plasma levels, such that the ratio of peak plasma levels to the plasma levels at 24 hours after administration, as well as the mean residence time of carvedilol, are within a desirable range for once-a-day therapy for the treatment and prophylaxis of cardiac and circulatory diseases.

An oral controlled release pharmaceutical composition for carvedilol that releases the carvedilol in a controlled manner so as to provide control over carvedilol plasma levels, such that the ratio of peak plasma levels to the plasma levels at 24 hours after administration, and the mean residence time of carvedilol, are within a desirable range for once-a-day therapy for the treatment and prophylaxis of cardiac and circulatory diseases, is thus required. Consequently, a method of providing control over carvedilol plasma levels in humans, said method consisting of orally administering to human subjects said oral controlled release pharmaceutical composition for once-a-day therapy for the treatment and prophylaxis of cardiac and circulatory diseases, would be possible. It has been found that the desired control over plasma levels for once-a-day therapy for the treatment and prophylaxis of cardiac and circulatory diseases is achieved by providing an oral controlled release pharmaceutical composition that provides a dissolution profile such that—

(a) Not more than 50% of the carvedilol is released after 2 hours;

(b) Not more than 70%, preferably between 25% and 70%, more preferably between 30% and 60% of the carvedilol is released after 4 hours;

(c) Not more than 90%, preferably between 50% and 90%, more preferably between 60% and 80% of the carvedilol is released after 8 hours; and

(d) Not less than 60%, preferably not less than 70% of the carvedilol is released after 12 hours;

when tested in United States Pharmacopoeia Type I apparatus using 0.1N HCl for 0-2 hours, and simulated intestinal fluid, pH 6.8, for 2-12 hours at rpm of 100.

### SUMMARY OF THE INVENTION

It is an object of the present invention to provide an oral controlled release pharmaceutical composition for once-a-day therapy for the treatment and prophylaxis of cardiac and circulatory diseases comprising carvedilol or its pharmaceutically acceptable salt or ester and release rate controlling excipients, wherein the said composition is adapted to release the carvedilol in a controlled manner so as to provide control over carvedilol plasma levels, such that the ratio of peak plasma levels to the plasma levels at 24 hours after administration, is within a desired range for once-a-day therapy for the treatment and prophylaxis of cardiac and circulatory diseases, preferably within 25:1 to 1:1, more preferably within 10:1 to 3:1, and still more preferably within 7:1 to 4:1; and the mean residence time of carvedilol is within a desirable range for once-a-day therapy for the treatment and prophylaxis of cardiac and circulatory diseases, preferably within about 10 to about 24 hours, more preferably within about 15 hours to about 20 hours.

Yet another object of the present invention is to provide a method of obtaining desired control over carvedilol plasma levels in humans for once-a-day therapy for the treatment and prophylaxis of cardiac and circulatory diseases by orally administering to human subjects said oral controlled release pharmaceutical composition.

The present invention provides an oral controlled release pharmaceutical composition for once-a-day therapy for the treatment and prophylaxis of cardiac and circulatory diseases comprising carvedilol or its pharmaceutically acceptable salt or ester and release rate controlling excipients, wherein the said composition is adapted to release the carvedilol in a controlled manner so as to provide a control over carvedilol plasma levels, such that the ratio of peak plasma levels to the plasma levels at 24 hours after administration, and the mean residence time of carvedilol, are within a desirable range for once-a-day therapy for the treatment and prophylaxis of cardiac and circulatory diseases.

The oral controlled release pharmaceutical composition of the present invention comprises carvedilol or its pharmaceutically acceptable salt or ester and a release rate controlling pharmaceutically acceptable excipient, such that carvedilol is released according to the following dissolution profile—

(a) Not more than 50% of the carvedilol is released after 2 hours;

(b) Not more than 70%, preferably between 25% and 70%, more preferably between 30% and 60% of the carvedilol is released after 4 hours;

(c) Not more than 90%, preferably between 50% and 90%, more preferably between 60% and 80% of the carvedilol is released after 8 hours; and

(d) Not less than 60%, preferably not less than 70% of the carvedilol is released after 12 hours;

when tested in vitro in United States Pharmacopoeia Type I apparatus using 0.1N HCl for 0-2 hours, and simulated intestinal fluid, pH 6.8, for 2-12 hours at rpm of 100.

The invention also relates to a method of obtaining a desired control over carvedilol plasma levels in humans for once-a-day therapy in the treatment and prophylaxis of cardiac and circulatory diseases, said method consisting of orally administering to human subjects an oral controlled release pharmaceutical composition comprising carvedilol or its pharmaceutically acceptable salt or ester and release rate controlling excipients, wherein the said composition is adapted to release the carvedilol in a controlled manner so as to provide a control over carvedilol plasma levels, such that the ratio of peak plasma levels to the plasma levels at 24 hours after administration, and the mean residence time of carvedilol, are within a desired range for the said once-a-day therapy for the treatment and prophylaxis of cardiac and circulatory diseases.

### BRIEF DESCRIPTION OF THE DRAWINGS

FIG. 1 shows the plasma concentration vs time profile obtained upon administration of one embodiment of the oral controlled release pharmaceutical composition of the present invention having 12.5 mg carvedilol, in comparison to that obtained for an equivalent dose of an immediate release composition.

### DETAILED DESCRIPTION OF THE INVENTION

The carvedilol or its pharmaceutically acceptable salt or ester may be used in the oral controlled release pharmaceutical composition of the present invention in the range of amounts equivalent to about 5 mg to about 100 mg of carvedilol. In particular, an oral controlled release pharmaceutical composition of the present invention may have carvedilol or its pharmaceutically acceptable salt or ester in an amount equivalent to 12.5 mg, 25 mg or 50 mg of carvedilol.

The oral controlled release pharmaceutical composition of the present invention releases the carvedilol in a controlled manner so as to provide a control over carvedilol plasma levels, such that the ratio of the peak plasma levels of carvedilol to the plasma levels at 24 hours after administration is in the range of 25:1 to 1:1, preferably in the range of 10:1 to 3:1, more preferably in the range of 7:1 to 4:1. The oral administration as referred to herein may be administration of the composition in the absence or presence of food, i.e. in the fasted mode or in the fed mode.

The oral controlled release pharmaceutical composition of the present invention is designed to increase the mean residence time of carvedilol in the body to a range from about 10 hours to about 24 hours, preferably about 15 hours to about 20 hours. The mean residence time is increased from about 3 to 4 times as compared to an immediate release composition. The half-life of carvedilol is increased by about 2 to 4 times as compared to the immediate release composition.

The oral controlled release pharmaceutical composition of the present invention comprises carvedilol or its pharmaceutically acceptable salt or ester and a release rate controlling excipient, such that the carvedilol is released according to the following dissolution profile—

(a) Not more than 50% of the carvedilol is released after 2 hours;

(b) Not more than 70% of the carvedilol is released after 4 hours;

(c) Not more than 90% of the carvedilol is released after 8 hours; and

(d) Not less than 60% of the carvedilol is released after 12 hours;

when tested in vitro in United States Pharmacopoeia Type I apparatus using 0.1N HCl for 0-2 hours, and simulated intestinal fluid, pH 6.8, for 2-12 hours at rpm of 100.

More particularly, the present invention provides an oral controlled release pharmaceutical composition comprising carvedilol or its pharmaceutically acceptable salt or ester and a release rate controlling excipient, wherein carvedilol is released according to the following dissolution profile:

(a) Not more than 50% of carvedilol is released after 2 hours;

(b) Between 25% and 70% of carvedilol is released after 4 hours;

(c) Between 50% and 90% of carvedilol is released after 8 hours; and

(d) Not less than 70% of carvedilol is released after 12 hours;

when tested in United States Pharmacopoeia Type I apparatus using 0.1N HCl for 0-2 hours, and simulated intestinal fluid, pH 6.8, for 2-12 hours at rpm of 100.

Still more particularly, the present invention provides an oral controlled release pharmaceutical composition comprising carvedilol or its pharmaceutically acceptable salt or ester and a release rate controlling excipient, wherein carvedilol is released according to the following dissolution profile:

(a) Not more than 50% of carvedilol is released after 2 hours;

(b) Between 30% to 60% of carvedilol is released after 4 hours;

(c) Between 60% to 80% of carvedilol is released after 8 hours;

(d) Not less than 70% of carvedilol is released after 12 hours;

when tested in United States Pharmacopoeia Type I apparatus using 0.1N HCl for 0-2 hours, and simulated intestinal fluid, pH 6.8, for 2-12 hours at rpm of 100.

The rate controlling excipient is any material that slows the rate of release of the drug from the dosage form. Usually, the rate controlling excipient is a polymer or a fatty compound or a mixture thereof. It may also comprise an ion-exchange resin. Examples of rate controlling polymers that may be used in the present invention include, but are not limited to:

cellulose ethers such as methylcellulose (MC), ethylcellulose (EC), hydroxyethylcellulose (HEC), hydroxypropyl cellulose (HPC), hydroxypropyl methylcellulose (HPMC), hydroxypropyl ethylcellulose (HPEC), carboxymethyl cellulose (CMC), crosslinked carboxymethyl cellulose (croscarmellose) and its alkali salts, ethylhydroxyethylcellulose (EHEC), hydroxyethyl methylcellulose (HEMC), hydrophobically modified hydroxyethyl cellulose (HMHEC), hydrophobically modified ethylhydroxyethylcellulose (HMEHEC), carboxymethyl hydroxyethylcellulose (CMHEC), carboxymethyl hydrophobically modified hydroxyethyl cellulose (CMHMHEC), and the like;

vinyl pyrrolidone polymers such as crosslinked polyvinylpyrrolidone or crospovidone, copolymers of vinyl pyrrolidone and vinyl acetate;

alkylene oxide homopolymers such as polypropylene oxide, preferably ethylene oxide homopolymers

a superdisintegrant polymer such as cross-linked polyvinylpyrrolidone, cross-linked sodium carboxymethylcellulose, carboxymethyl starch, sodium carboxymethyl starch, potassium methacrylate-divinylbenzene copolymer, polyvinyl alcohols, amylose, cross-linked amylose, starch derivatives, microcrystalline cellulose and cellulose derivatives, alpha-, beta-and gamma-cyclodextrin and dextrin derivatives such as cross-linked carboxymethylcellulose

gums of plant, animal, mineral or synthetic origin such as (i) agar, alginates, carrageenan, furcellaran derived from marine plants, (ii) guar gum, gum arabic, gum tragacanth, karaya gum, locust bean gum, pectin derived from terrestrial plants, (iii) microbial polysaccharides such as dextran, gellan gum, rhamsan gum, welan gum, xanthan gum, and (iv) synthetic or semi-synthetic gums such as propylene glycol alginate, hydroxypropyl guar and modified starches like sodium starch glycolate, and the like; and

an acrylic acid polymer such as cross-linked polymer available under the trade name Carbopol® or homopolymers and co-polymers of acrylate or methacrylate monomers for example polymethacrylates marketed under the brand names of Eudragit®, particularly Eudragit® RS and Eudragit® RL.

Examples of fatty compounds that may be used as the rate controlling excipients in the present invention include various waxes such as digestible, long chain (C₈-C₅₀, especially C₁₂-C₄₀), substituted or unsubstituted hydrocarbons, such as fatty acids, fatty alcohols, glyceryl esters of fatty acids, mineral and vegetable oils and waxes. Hydrocarbons having a melting point of between 25° and 90° C. are preferred. Of these long chain hydrocarbon materials, fatty (aliphatic) alcohols are preferred.

In one embodiment of the present invention, the release rate controlling excipient is a hydrophilic swellable polymer. In a preferred embodiment the hydrophilic swellable polymer is polyethylene oxide. Polyethylene oxide is a nonionic homopolymer of ethylene oxide, containing 2000 to over 100,000 repeating oxyethylene groups. The molecular weight of polyethylene oxide ranges between 100,000 Daltons and 7,000,000 Daltons. It is commercially available as Polyox® from Union Carbide. The higher molecular weight polyethylene oxide grades (molecular weight 3,000,000 to 7,000,000 Daltons), such as Polyox® WSR coagulant with an approximate molecular weight of 5,000,000 Daltons, are used in more preferred embodiments of the present invention to provide delayed, sustained or controlled drug release. The polymer swells upon contact with aqueous fluid from the environment of use to form a hydrophilic gel matrix. This matrix expands with time and causes diffusion of the drug at a predetermined rate, depending upon the concentration and grade of the polymer used. In a more preferred embodiment, Polyox® WSR coagulant is used as the swelling agent in a concentration from about 20% to about 60% by weight of the tablet.

The pharmaceutical composition of this embodiment may also include various pharmaceutically acceptable excipients, for example wicking agents such as microcrystalline cellulose; disintegrants such as starch, cellulose derivatives, gums, crosslinked polymers and the like; binders such as starch, gelatin, sugars, cellulose derivatives, polyvinyl pyrrolidone and the like; lubricants such as talc, magnesium stearate, colloidal silicon dioxide, polyethylene glycol and mixtures thereof.

In one embodiment of the present invention, microcrystalline cellulose is present as a wicking agent. The microcrystalline cellulose is dispersed in the matrix of the hydrophilic swellable polymer, preferably polyethylene oxide.

The oral controlled release pharmaceutical composition of the present invention may be in the form of a matrix formulation, a coated composition, an ion exchange composition, an osmotic system comprising a core covered with a semipermeable membrane, and various other controlled release compositions known to a person skilled in the art.

A matrix formulation for the present invention comprises a core comprising carvedilol and a release rate controlling excipient, preferably a hydrophilic swellable polymer, more preferably polyethylene oxide, and particularly preferably a polyethylene oxide having a molecular weight of 5,000,000 Daltons.

A coated composition that provides a controlled release of carvedilol is obtained by coating a drug containing core with release rate controlling excipients, using techniques known to a person skilled in the art. The osmotic system for the controlled release of carvedilol comprises a core comprising the drug and other pharmaceutically acceptable excipients, covered with a semipermeable membrane, the membrane having an orifice for the release of carvedilol in a controlled manner over a defined period of time.

The matrix formulations containing release rate controlling excipients may be prepared by mixing carvedilol or its pharmaceutically acceptable salt or ester, with a release rate controlling excipient. A controlled release pharmaceutical composition may also be obtained by coating particles, pellets, granules or tablets of carvedilol with release rate controlling excipients, such as hardened gelatin, methyl cellulose, ethyl cellulose, methacrylates such as anionic polymer of methacrylic acid and methacrylates with a carboxylic group, cationic polymer with a dimethylaminoethyl ammonium group, copolymers of acrylates and methacrylates with quarternary ammonium group in combination with sodium carboxymethylcellulose, copolymers of acrylate and methacrylates with quarternary ammonium group and the like, commercially available as Eudragit®, for example Eudragit® RS, Eudragit® RL, Eudragit® L, Eudragit® E, Eudragit® S, Eudragit® RD and the like; hydroxypropyl cellulose, polyvinyl acetate, polyvinyl acetate phthalate, shellac, various waxes and the like.

In one embodiment of the present invention the oral controlled release pharmaceutical composition is obtained in the form of a tablet comprising carvedilol, a swelling agent as the release rate controlling excipient and other pharmaceutically acceptable excipients. The swelling agent that may be used in this embodiment may be selected from above-mentioned release rate controlling excipients such as cellulose ethers, vinylpyrrolidone polymers, alkylene oxide homopolymers, superdisintegrant polymers, natural gums, and acrylic polymers.

In another embodiment, the oral controlled release pharmaceutical composition of the present invention is obtained in the form of an oral osmotic controlled drug delivery system comprising a core comprising carvedilol, a polymeric swelling agent consisting of one or more swellable hydrophilic polymers, water soluble compounds for inducing osmosis, and other pharmaceutical excipients; the core being surrounded by a semi-permeable membrane having a passageway for the release of carvedilol. Examples of swellable hydrophilic polymers that may be used in this embodiment include cellulose derivatives, vinyl pyrrolidone polymers such as crosslinked polyvinylpyrrolidone or crospovidone, copolymers of vinyl pyrrolidone and vinyl acetate, and gums of natural and synthetic origin. A combination of xanthan gum and crosslinked sodium carboxymethyl cellulose is used as the preferred polymeric swelling agent in this embodiment in an amount ranging from about 5% to about 10% by weight of the core. Water soluble compounds used for inducing osmosis may include one or more pharmaceutically acceptable and pharmacologically inert water-soluble compounds referred to in the pharmacopoeias such as United States Pharmacopoeia, as well as in Remington: The Science and Practice of Pharmacy, edition 20; Lippincott Williams and Wilkins, Philadelphia (2000), and are used in an amount ranging from about 10% to about 50% by weight of the core. One or more types of cellulose acetates may be used along with plasticisers to form the semi-permeable wall. The passageway comprises of orifices, bores or apertures and the like, through the semipermeable wall prepared by various methods such as those disclosed in U.S. Pat. No. 3,916,899.

One embodiment of the pharmaceutical composition of the present invention may comprise the steps of mixing carvedilol or its pharmaceutically acceptable salt or ester with the release rate controlling and other pharmaceutically acceptable excipients and forming a pharmaceutical dosage form by conventional means. In an alternative embodiment, a core may be formed from the mixture of carvedilol or its pharmaceutically acceptable salt or ester and the pharmaceutically acceptable excipients, which may or may not include a rate controlling excipient; and then the core may be coated by conventional methods with a coating composition comprising the rate controlling excipient. The pharmaceutical dosage form may be formed by any of the various methods known in the art. It may be formed into capsules by filling the mixture of carvedilol or its pharmaceutically acceptable salt or ester and pharmaceutically acceptable excipients into capsules. Alternatively, the mixture may be formed into granules or pellets by conventional means such as dry granulation, wet granulation, extrusion, spheronisation and the like. The granules or pellets may be filled into capsules or may be compressed into tablets.

In one specific embodiment, the oral controlled release pharmaceutical composition may be in the form of an oral osmotic controlled drug delivery system. The oral osmotic controlled drug delivery system for carvedilol may be obtained by mixing carvedilol with the polymeric swelling agent and the water-soluble osmosis inducing agents, and the mixture is granulated using a solution of a binder. The granules are dried and mixed with lubricants, followed by compression of the lubricated mass to obtain the core, using conventional procedures known to a person skilled in the art. A solution of cellulose acetate and a plasticiser in a suitable solvent is then used to form the semi-permeable membrane. The solution of the cellulose acetate is loaded on the core to a desirable weight gain using conventional coating techniques known to a person skilled in the art. A passageway is then introduced in the semi-permeable membrane using mechanical or laser drilling.

In another embodiment, the oral controlled release pharmaceutical composition for carvedilol may be obtained by mixing carvedilol with ethyl cellulose to obtain a dry powder blend. This blend is mixed with isopropanol and the wet mass is passed through a #20 sieve to obtain granules. The granules are dried in a fluid bed drier at 50° C. and again passed through a suitable sieve to remove the fines. These granules are then coated with a solution comprising ethyl cellulose, HPMC, dibutyl phthalate and talc, using a suitable solvent system, in a fluid bed coater. The granules are coated to a weight gain of about 15% to about 20% of their weight. The dry granules are either encapsulated, or compressed on a rotary compression machine to obtain tablets.

The oral controlled release pharmaceutical composition as herein described, is orally administered to humans to provide desired control over carvedilol plasma levels in humans for once-a-day therapy for the treatment and prophylaxis of cardiac and circulatory diseases. The oral controlled release pharmaceutical composition may be administered to the patient on an empty stomach or with meals.

The examples that follow do not limit the scope of the invention and are presented as illustrations.

### EXAMPLE 1

This example illustrates one embodiment of the pharmaceutical composition of the present invention and a process for its preparation. Tablets were prepared according to the formula given in Table 1 below.

TABLE 1

| | | Quantity |
|---|---|---|
| Sr. | | (Percent weight |
| No. | Ingredients | of the tablet) |
| 1. | Carvedilol | 5.95 |
| 2. | Polyethylene oxide (Polyox ® WSR Coagulant) | 38.095 |
| 3. | Microcrystalline cellulose | 28.57 |
| 4. | Starch | 17.62 |
| 5. | Polyvinyl pyrrolidone (PVP K-30) | 4.76 |
| 6. | Talc | 2.38 |
| 7. | Magnesium stearate | 1.43 |
| 8. | Colloidal silicon dioxide (Aerosil ® 200) | 1.19 |

Carvedilol, microcrystalline cellulose and starch were dry blended in the amounts mentioned in Table 1 above, after passing the individual ingredients through a #60 sieve (as defined by American Society for Testing and Materials, ASTM). Polyethylene oxide, passed through a #20 sieve (as defined by American Society for Testing and Materials, ASTM), was then added to this dry powder blend. PVP K-30 dissolved in a sufficient quantity of isopropanol was used to granulate the dry powder blend. The wet mass was passed through a #20 sieve to obtain granules of the formulation. The granules were dried in a fluid bed drier and the dried granules were passed through a #20 sieve again to remove fines. A mixture of talc, magnesium stearate and Aerosil® 200, passed through a #60 sieve, was then used to lubricate the dry granules. This lubricated mass was then compressed using 7 mm standard concave punches to obtain the final tablets.

The tablets so obtained were subjected to dissolution testing using United States Pharmacopoeia Type I dissolution apparatus at 100 rpm. The dissolution medium used was 900 ml of 0.1N HCl for 0-2 hours, and 900 ml of simulated intestinal fluid, pH 6.8, for 2-12 hours. The results of the dissolution test are mentioned in Table 2 below.

TABLE 2

| Time (hours) | % drug released (±SD) |
|---|---|
| 2 | 25.24 ± 3.03 |
| 4 | 39.77 ± 3.19 |
| 8 | 72.13 ± 6.66 |
| 12 | 87.98 ± 4.80 |

### EXAMPLE 2

This example illustrates another embodiment of the pharmaceutical composition of the present invention and a process for its preparation. A controlled release formulation was made as per the formula given in Table 3 below.

TABLE 3

| | | Quantity (Percent |
|---|---|---|
| Sr. No. | Ingredients | weight of the tablet) |
| 1. | Carvedilol | 81.74 |
| 2. | Ethyl cellulose N 50 | 14.99 |
| 3. | Hydroxypropyl methylcellulose (HPMC | 1.09 |
| | ES) | |
| 4. | Dibutyl phthalate | 1.09 |
| 5. | Talc | 1.09 |
| | Total | 100.0 |

Carvedilol was mixed with a part of the ethyl cellulose and granulated with isopropanol. The wet mass was passed through a #20 sieve to obtain the granules, which were dried in a fluid bed drier at 50° C., and again sifted on drying to remove the fines. These granules were then coated in a fluid bed coater with a solution of the remaining amount of ethyl cellulose, hydroxypropyl methylcellulose, dibutyl phthalate and talc, in a suitable solvent system, to a defined weight gain.

### EXAMPLE 3

This example illustrates the pharmaceutical composition of the present invention in the form of oral osmotic controlled release tablets and a process for its preparation. Oral osmotic controlled release tablets of carvedilol were prepared according to the formula given in Table 4 below.

TABLE 4

| | | Quantity (Percent |
|---|---|---|
| Sr. No. | Ingredients | weight of the core) |
| Core | | |
| 1. | Carvedilol | 8.0 |
| 2. | Sodium chloride | 35.83 |
| 3. | Mannitol | 35.83 |
| 4. | Xanthan gum | 7.04 |
| 5. | Croscarmellose sodium (Ac-Di-Sol) | 7.04 |
| 6. | Yellow oxide of iron | 0.16 |
| 7. | Red oxide of iron | 0.16 |
| 8. | Polyvinyl pyrrolidone (PVP K30) | 2.24 |
| 9. | Talc | 1.92 |
| 10. | Magnesium stearate | 0.96 |
| 11. | Colloidal silicon dioxide | 0.83 |
| Coat - | | |
| 1. | Cellulose acetate | 4.1 |
| 2. | Polyethylene glycol (PEG 3350) | 0.65 |

Carvedilol, sodium chloride, mannitol, xanthan gum, Ac-Di-Sol, yellow oxide of iron and red oxide of iron were mixed and passed through a #60 sieve (as defined by ASTM), and granulated using a solution of PVP K-30 in isopropyl alcohol. The granules so obtained were passed through a #20 sieve (as defined by ASTM) and dried. Talc, magnesium stearate and colloidal silicon dioxide were mixed and passed through a #60 sieve. This mixture was then mixed with the dried granules. The lubricated mixture was then compressed to obtain the cores. A solution of cellulose acetate and polyethylene glycol (PEG 3350) in acetone was used to coat the cores to a weight gain of 5%. An orifice was then drilled in the coated tablets using laser drilling.

The tablets so obtained were subjected to dissolution testing using United States Pharmacopoeia Type I dissolution apparatus at 100 rpm. The dissolution medium used was 900 ml of 0.1N HCl for 0-2 hours, and 900 ml of simulated intestinal fluid, pH 6.8, for 2-12 hours. The results of the dissolution test are mentioned in Table 5 below.

TABLE 5

| Time (hours) | % drug released (±SD) |
|---|---|
| 2 | 24.55 ± 0.94 |
| 4 | 43.45 ± 2.62 |
| 8 | 66.65 ± 0.67 |
| 12 | 73.98 ± 0.48 |

### EXAMPLE 4

In another embodiment of the present invention, oral osmotic controlled release pharmaceutical tablets for carvedilol were obtained according to the formula given in Table 6 below.

TABLE 6

| | | Quantity (Percent |
|---|---|---|
| Sr. No. | Ingredients | weight of the core) |
| Core | | |
| 1. | Carvedilol | 8.92 |
| 2. | Sodium chloride | 28.57 |
| 3. | Mannitol | 28.57 |
| 4. | Xanthan gum | 13.57 |
| 5. | Croscarmellose sodium (Ac-Di-Sol) | 13.57 |
| 6. | Yellow oxide of iron | 0.35 |
| 7. | Polyvinyl pyrrolidone (PVP K30) | 3.21 |
| 8. | Talc | 2.5 |
| Coat - | | |
| 1. | Cellulose acetate | 4.1 |
| 2. | Polyethylene glycol (PEG 3350) | 0.65 |

The tablets were prepared according to the procedure given in Example 3 above. The tablet so obtained were subjected to dissolution testing using United States Pharmacopoeia Type I dissolution apparatus at 100 rpm. The dissolution medium used was 900 ml of 0.1N HCl for 0-2 hours, and 900 ml of simulated intestinal fluid, pH 6.8, for 2-12 hours. The results of the dissolution test are mentioned in Table 7 below.

TABLE 7

| Time (hours) | % drug released (±SD) |
|---|---|
| 2 | 23.15 ± 3.40 |
| 4 | 40.42 ± 2.94 |
| 8 | 65.88 ± 11.36 |
| 12 | 76.23 ± 8.94 |

### EXAMPLE 5

The bioavailability of the controlled release carvedilol formulation (12.5 mg tablet, Example 1) of the present invention and that of conventional immediate release carvedilol formulation (2×6.25 mg) were studied. A single-dose, open label, randomized, comparative and two-way crossover study, with a seven day washout period, was undertaken for the same. Cardivas (Sun Pharma, Lot no. JK10381, Exp. Date: March 2003) 12.5 mg (2×6.25 mg tablets) was used as the immediate release carvedilol formulation.

The pharmacokinetic assessment was based on the plasma levels of carvedilol measured by blood sampling. Blood samples were obtained before dosing and at the following times after administration of both the reference and test medications—0.5, 1.5, 2, 2.5, 3, 3.5, 4, 5, 6, 8, 10, 12, 16, 24, 28 and 32 hours.

Six healthy male volunteers were enrolled for the study and all of them completed the two-way crossover study. The subjects were fasted overnight before dosing and for 4 hours thereafter. Drinking water was prohibited 2 hours before dosing and 2 hours thereafter, but was allowed ad lib at all other times. Standard meals were provided at 4 hours and 8 hours after dosing and at appropriate times thereafter. Meal plans were identical for both the periods.

Subjects received a single controlled release tablet of carvedilol (12.5 mg, Example 1) with 240 ml of water at ambient temperature after the overnight fast, as the test medication, while the reference medication was administered as two tablets of Cardivas (Sun Pharma), each of 6.25 mg.

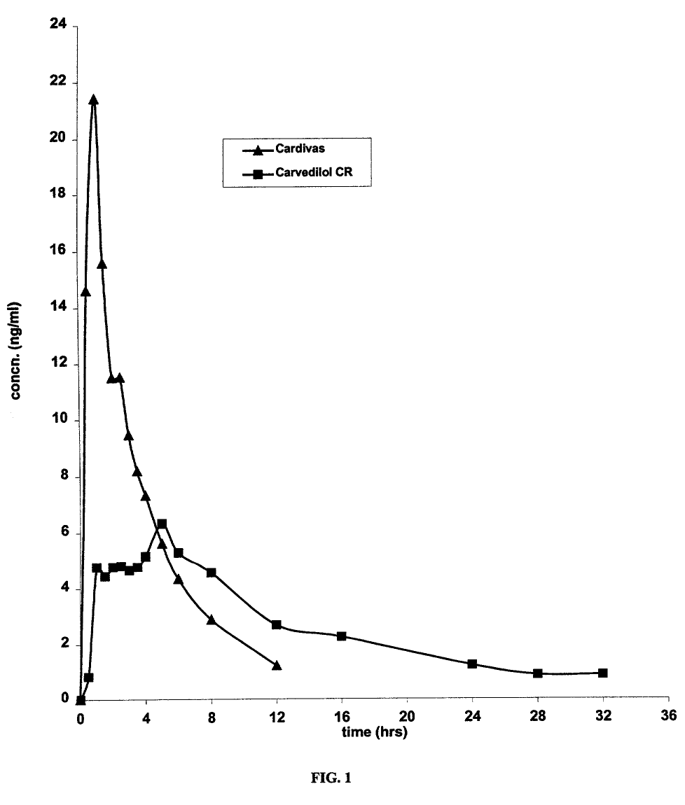

The plasma concentration of carvedilol was determined for samples collected at different time points and averaged over the six volunteers. The data is given in Table 8 below. The plasma concentration versus time profile is illustrated in FIG. 1.

TABLE 8

| | Plasma concentration (ng/ml) (Mean ± SD) | |
|---|---|---|
| | Carvedilol controlled release | Cardivas (Sun Pharma, |
| Time (hrs) | tablet (12.5 mg) | 2 × 6.25 mg) |
| 0.5 | 0.82 ± 1.49 | 14.61 ± 5.60 |
| 1.0 | 4.77 ± 5.08 | 21.42 ± 13.57 |
| 1.5 | 4.45 ± 1.51 | 15.59 ± 7.66 |
| 2.0 | 4.78 ± 1.85 | 11.49 ± 8.19 |
| 2.5 | 4.82 ± 1.92 | 11.51 ± 5.62 |
| 3.0 | 4.66 ± 2.08 | 9.47 ± 3.95 |
| 3.5 | 4.78 ± 2.16 | 8.19 ± 3.70 |
| 4.0 | 5.16 ± 2.99 | 7.32 ± 3.12 |
| 5.0 | 6.32 ± 3.53 | 5.61 ± 3.40 |
| 6.0 | 5.29 ± 2.23 | 4.34 ± 2.19 |
| 8.0 | 4.56 ± 2.08 | 2.88 ± 1.88 |
| 12.0 | 2.68 ± 1.55 | 1.21 ± 1.15 |
| 16.0 | 2.25 ± 1.35 | — |
| 24.0 | 1.22 ± 0.88 | — |

For the controlled release tablets, the ratio of peak plasma level to the plasma level at 24 hours after administration, when calculated from the above averaged plasma concentration, was about 5.2. The ratio could not be determined for the immediate release formulation but it would be obvious that the ratio would be comparatively very large, perhaps 10 to 20 times in magnitude, as compared to the controlled release tablets.

The ratios of peak plasma level to the plasma level at 24 hours after administration were also calculated from the subjects' individual plasma data. These ratios were 9.0, 4.0, 5.6, 9.2 and 5.26 for five of the subjects; the sixth subject showed comparatively low bioavailability and carvedilol concentrations below the sensitivity limits of the assay. The mean ±standard deviation obtained for the five values of the ratios was 6.6±2.36.

Half-life was determined from the individual subject plasma data and the values obtained are given in Table 9 below.

TABLE 9

| | Carvedilol controlled release | Cardivas (Sun Pharma, |
|---|---|---|
| Subject | tablet (12.5 mg) | 2 × 6.25 mg) |
| 1 | 5.80 hr | 2.04 hr |
| 2 | 13.69 hr | 3.50 hr |
| 3 | 11.66 hr | 5.37 hr |
| 4 | 19.19 hr | 10.25 hr |
| 5 | 3.01 hr | 3.02 hr |
| 6 | 7.92 hr | 3.71 hr |
| Mean ± | 10.21 ± 5.86 hr | 4.65 ± 2.95 hr |
| S.D | | |

The other pharmacokinetic parameters calculated include area under the curve (AUC_{α}) and area under the moment curve (AUMC_{α}). The AUC is calculated using the formula:

$${AUC}_{α} = \sum {C}_{avg} \times \frac{Δ t + {C}_{last}}{{λ}_{n}}$$

The AUMC is calculated using the formula:

$${AUMC}_{α} = ( \sum {( Ct )}_{avg} \times Δ t ) + ( \frac{{C}_{last} \times {t}_{last}}{{λ}_{n}} ) + ( \frac{{C}_{last}}{{λ}_{n}^{2}} )$$

The AUC_{α} and AUMC_{α} values obtained were used to calculate the mean residence time for the controlled release formulation of Example 1, and the immediate release formulation used as the reference. The mean residence time (MRT) was calculated using the formula:

$$MRT = \frac{{AUMC}_{α}}{{AUC}_{α}}$$

The mean residence time for the controlled release formulation of Example 1 was found to be 16.02±6.73 hours, as compared to 5.50±2.76 hours for the immediate release formulation.

While the invention has been described with reference to specific embodiments, this was done for purposes of illustration only and should not be considered to limit the spirit or the scope of the invention.

What is claimed is:

1. An oral controlled release pharmaceutical composition for once-a-day therapy for the treatment and prophylaxis of cardiac and circulatory diseases comprising carvedilol or its pharmaceutically acceptable salt or ester and release rate controlling excipients, wherein the said composition is adapted to release the carvedilol in a controlled manner so as to provide control over carvedilol plasma levels, such that the ratio of peak plasma levels to the plasma levels at 24 hours after administration, and the mean residence time of carvedilol, are within a desirable range for said once-a-day therapy for the treatment and prophylaxis of cardiac and circulatory diseases.

2. An oral controlled release pharmaceutical composition as claimed in claim 1, wherein the amount of carvedilol or its pharmaceutically acceptable salt or ester expressed as carvedilol, is in the range from about 5 mg to about 100 mg.

3. An oral controlled release pharmaceutical composition as claimed in claim 2 wherein the ratio of peak carvedilol plasma levels to carvedilol plasma levels at 24 hours, after oral administration to human subjects, is in the range of 25:1 to 1:1.

4. An oral controlled release pharmaceutical composition as claimed in claim 3 wherein the ratio of peak carvedilol plasma levels to carvedilol plasma levels at 24 hours, after oral administration to human subjects, is in the range of 10:11 to 3:1.

5. An oral controlled release pharmaceutical composition as claimed in claim 4 wherein the ratio of peak carvedilol plasma levels to carvedilol plasma levels at 24 hours, after oral administration to human subjects, is in the range of 7:1 to 4:1.

6. An oral controlled release pharmaceutical composition as claimed in claim 1 wherein the mean residence time of carvedilol is in the range of about 10 hours to about 24 hours.

7. An oral controlled release pharmaceutical composition as claimed in claim 6 wherein the mean residence time of carvedilol is in the range of about 15 hours to about 20 hours.

8. An oral controlled release pharmaceutical composition as claimed in claim 1 wherein the mean residence time of carvedilol is increased by about 3 to 4 times as compared to the immediate release composition.

9. An oral controlled release pharmaceutical composition as claimed in claim 1 wherein the half-life of carvedilol is increased by about 2 to 4 times as compared to the immediate release composition.

10. An oral controlled release pharmaceutical composition as claimed in claim 1 wherein the release rate controlling pharmaceutically acceptable excipient is a hydrophilic swellable polymer.

11. An oral controlled release pharmaceutical composition as claimed in claim 1 wherein the release rate controlling pharmaceutically acceptable excipient is a water insoluble polymer.

12. An oral controlled release pharmaceutical composition as claimed in claim 10 wherein the hydrophilic swellable polymer is polyethylene oxide (PEO).

13. An oral controlled release pharmaceutical composition as claimed in claim 12 wherein the polyethylene oxide has molecular weight in the range from 3,000,000 Daltons to 7,000,000 Daltons.

14. An oral controlled release pharmaceutical composition as claimed in claim 13 wherein the polyethylene oxide has molecular weight of 5,000,000 Daltons.

15. An oral controlled release pharmaceutical composition as claimed in claim 12 wherein microcrystalline cellulose is present as a wicking agent.

16. An oral controlled release pharmaceutical composition as claimed in claim 1 wherein the said composition is in the form of an oral osmotic delivery system comprising:
a. a core comprising carvedilol, a polymeric swelling agent, one or more water-soluble compounds for inducing osmosis, and optionally other pharmaceutical excipients;
b. a semi-permeable membrane surrounding the core (a), which is permeable to the surrounding fluid but impermeable to the contents of the core; and
c. a passageway through the membrane (b) for releasing the contents of the core.

17. An oral controlled release pharmaceutical composition as claimed in claim 16 wherein the polymeric swelling agent comprises one or more swellable hydrophilic polymers selected from the group consisting of cellulose derivatives, vinyl pyrrolidone polymers such as crosslinked polyvinylpyrrolidone, copolymers of vinyl pyrrolidone and vinyl acetate, and gums of natural and synthetic origin.

18. An oral controlled release pharmaceutical composition as claimed in claim 17 wherein the polymeric swelling agent comprises a mixture of sodium carboxymethyl cellulose and xanthan gum in a 1:1 ratio.

19. An oral controlled release pharmaceutical composition as claimed in claim 1 comprising carvedilol or its pharmaceutically acceptable salt or ester and a release rate controlling excipient, such that the carvedilol is released according to the following dissolution profile—
a. Not more than 50% of carvedilol is released after 2 hours;
b. Not more than 70% of carvedilol is released after 4 hours;
c. Not more than 90% of carvedilol is released after 8 hours; and
d. Not less than 60% of carvedilol is released after 12 hours;
when tested in vitro in United States Pharmacopoeia Type I apparatus using 0.1N HCl for 0-2 hours, and simulated intestinal fluid, pH 6.8, for 2-12 hours at rpm of 100.

20. An oral controlled release pharmaceutical composition as claimed in claim 19 wherein carvedilol is released as per the following dissolution profile—
a. Not more than 50% of carvedilol is released after 2 hours;
b. Between 25% and 70% of carvedilol is released after 4 hours;
c. Between 50% and 90% of carvedilol is released after 8 hours; and
d. Not less than 70% of carvedilol is released after 12 hours;
when tested in United States Pharmacopoeia Type I apparatus using 0.1N HCl for 0-2 hours, and simulated intestinal fluid, pH 6.8, for 2-12 hours at rpm of 100.

21. An oral controlled release pharmaceutical composition as claimed in claim 20 wherein carvedilol is released as per the following dissolution profile—
a. Not more than 50% of carvedilol is released after 2 hour;
b. Between 30% and 60% of carvedilol is released after 4 hours;
c. Between 60% and 80% of carvedilol is released after 8 hours; and
d. Not less than 70% of carvedilol is released after 12 hours;
when tested in United States Pharmacopoeia Type I apparatus using 0.1N HCl for 0-2 hours, and simulated intestinal fluid, pH 6.8, for 2-12 hours at rpm of 100.

22. A method of obtaining desired control over carvedilol plasma levels in humans for once-a-day therapy for the treatment and prophylaxis of cardiac and circulatory diseases, said method consisting of orally administering to human subjects an oral controlled release pharmaceutical composition comprising carvedilol or its pharmaceutically acceptable salt or ester and release rate controlling excipients, the said composition releasing the carvedilol in a controlled manner so as to provide control over carvedilol plasma levels, such that the ratio of peak plasma levels to the plasma levels at 24 hours after administration, and the mean residence time of carvedilol, are within a desirable range for said once-a-day therapy for the treatment and prophylaxis of cardiac and circulatory diseases.

23. A method as claimed in claim 22 wherein the ratio of peak carvedilol plasma levels to carvedilol plasma levels at 24 hours, after oral administration to human subjects, is in the range of 25:1 to 1:1.

24. A method as claimed in claim 23 wherein the ratio of peak carvedilol plasma levels to carvedilol plasma levels at 24 hours, after oral administration to human subjects, is in the range of 10:1 to 3:1.

25. A method as claimed in claim 24 wherein the ratio of peak carvedilol plasma levels to carvedilol plasma levels at 24 hours, after oral administration to human subjects, is in the range of 7:1 to 4:1.

26. A method as claimed in claim 22 wherein the mean residence time of carvedilol is in the range of about 10 hours to about 24 hours.

27. A method as claimed in claim 26 wherein the mean residence time of carvedilol is in the range of about 15 hours to about 20 hours.

28. A method as claimed in claim 22 wherein the mean residence time of carvedilol is increased by about 3 to 4 times as compared to the immediate release composition.

29. A method as claimed in claim 22 wherein the half-life of carvedilol is increased by about 2 to 4 times as compared to the immediate release composition.

30. A method as claimed in claim 22 comprising carvedilol or its pharmaceutically acceptable salt or ester and a release rate controlling pharmaceutically acceptable excipient, such that the carvedilol is released according to the following dissolution profile—
a. Not more than 50% of carvedilol is released after 2 hours;
b. Not more than 70% of carvedilol is released after 4 hours;
c. Not more than 90% of carvedilol is released after 8 hours; and
d. Not less than 60% of carvedilol is released after 12 hours;
when tested in vitro in United States Pharmacopoeia Type I apparatus using 0.1N HCl for 0-2 hours, and simulated intestinal fluid, pH 6.8, for 2-12 hours at rpm of 100.

31. A method as claimed in claim 30 wherein carvedilol is released as per the following dissolution profile—
a. Not more than 50% of carvedilol is released after 2 hours;
b. Between 25% and 70% of carvedilol is released after 4 hours;
c. Between 50% and 90% of carvedilol is released after 8 hours; and
d. Not less than 70% of carvedilol is released after 12 hours;
when tested in United States Pharmacopoeia Type I apparatus using 0.1N HCl for 0-2 hours, and simulated intestinal fluid, pH 6.8, for 2-12 hours at rpm of 100.

32. A method as claimed in claim 31 wherein carvedilol is released as per the following dissolution profile—
a. Not more than 50% of carvedilol is released after 2 hour;
b. Between 30% and 60% of carvedilol is released after 4 hours;
c. Between 60% and 80% of carvedilol is released after 8 hours; and
d. Not less than 70% of carvedilol is released after 12 hours;
when tested in United States Pharmacopoeia Type I apparatus using 0.1N HCl for 0-2 hours, and simulated intestinal fluid, pH 6.8, for 2-12 hours at rpm of 100.
